(19) United States
(12) Patent Application Publication
(10) Pub. No.: US 2006/0129029 A1
(43) Pub. Date: Jun. 15, 2006

(54) Method and surgical tool to assist trocar inserting during surgery

(76) Inventor: Anatole Menon-Johannsson, London (GB)

Correspondence Address: BARNES & THORNBURG, LLP, P.O. BOX 2786, CHICAGO, IL, 60690-2786, US

(21) Appl. No.: 10/543,790

(22) PCT Filed: Jan. 27, 2004
(86) PCT No.: PCT/GB04/00340

§ 371 Date: Oct. 11, 2005

(51) Int. Cl. A61B 1/00

(52) U.S. Cl. 600/104

(57) ABSTRACT

A method of identifying a position at which an opening can be made in the peritoneum of an anaesthetised patient to enable the insertion of a laparoscopic tool to assist in laparoscopic surgery after an initial umbilical opening has been made. The method involves the steps of: inflating the abdomen via the umbilical opening or otherwise lifting the abdomen, selecting an elongate tool having a trocar support ring (1) at its leading end, introducing the ring ahead of the tool through the umbilical opening, and pushing the tool in a forward sense so as to press the ring against the peritoneum to deform and locally stretch the skin, and thereafter viewing the deformation externally. The pointed end of a trocar is then positioned centrally of the deformation of the abdominal wall and forced through the area of stretched skin so that the tip of the trocar penetrates the skin and is received in the support ring. An elongate tool for use in the method includes a loop or ring which extends from one end of the tool with its plane non-parallel to the axis of the tool. The loop or ring and the body of the tool are substantially rigid and non-deformable to allow the loop or ring to be pushed into contact with the internal abdomen wall and to stretch the skin of the wall to form an externally visible taut area of skin. The size of the loop or ring is selected so that it is to small to allow the trocar to pass completely therethrough, and optionally a cage or cup is located to the rear of the loop or ring to receive the leading end of a trocar thereby to prevent the trocar point from penetrating an internal organ. The loop or ring is discontinuous and defines a U or C shape.

### FIELD OF THE INVENTION

This invention concerns a device and method to assist in the insertion of laparoscopic trocars.

### BACKGROUND TO THE INVENTION

Minimally invasive surgery creates significant benefits for the patient and the health care delivery service. Patients have fewer post-operative complications plus a speedier discharge following such procedures, whilst hospitals are able to safely increase their turnover of patients because of these shorter hospital stays.

Laparoscopic surgery is being used to treat an increasing number of conditions in a range of specialties, including Gynaecology, General surgery, Cardio-thoracic surgery and Orthopaedics.

Laparoscopic surgery involves the following stages:

Anaesthetise the patient

Umbilical tool and then port insertion

Insufflation of the abdomen with gas or physically lifting up the abdominal wall

Insertion of telescope

Insertion of further abdominal tools

Performance of surgical procedure

Removal of tools

Suturing of abdominal wounds

The least elegant part of laparoscopic procedures is the insertion of second and subsequent abdominal tools. These have to be preceded with a trocar insertion, possibly close to an internal organ, which could be damaged if impaled by the trocar.

The procedure normally involves the surgeon viewing the interior of the abdominal wall via the laparoscope as the second trocar is inserted. Insertion involves pressure and twisting of the trocar in order to incise layers of tissue, and the final stages of insertion are heralded by peritoneal membrane tenting before the trocar tip breaches the abdominal wall and enters the abdominal cavity. At least in the latter stages of insertion, the surgeon will try to angle the trocar away from vulnerable organs, to avoid inadvertent perforation.

The procedure is relatively straightforward when the surgeon is experienced, the patient is of average size, and the surgeon is using new sharp trocars. However, in less experienced hands, with older tools, and small or obese patients, problems can be experienced.

It is an object of the present invention to provide a device and method for inserting a trocar in a surgical procedure so as to reduce the pressure needed to insert the trocar and to reduce potential damage to internal organs.

It has been proposed to provide a tool attached to the end of a laparoscopic telescope, to project ahead of the viewing lens of the telescope, the tool having a ring at its foremost end, which can be pushed into contact with the abdomen wall at a point where a trocar is to be inserted, for supporting the abdominal wall around the entry point of the trocar.

Typically the plane of the ring is angled relative to the axis of the telescope, and typically the telescope has a 30° oblique visual field so that the ring can be positioned to as to appear in the top of the field of view, as seen by the surgeon through the telescope.

Hitherto is has been proposed to view the peritoneum through the telepscope and in use the trocar has first been pushed gently against the abdomen where an incision is to be made and the ring has been positioned on the inside of the abdominal wall adjacent to the peritoneum so as to surround the protrusion in the peritoneum caused by the point of the trocar as it is pressed against the external surface of the abdomen wall. The trocar has then been rotated and forced inwardly in known manner to form the incision.

### SUMMARY OF THE INVENTION

Acording to one aspect of the present invention there is provided a method of identifying a position at which an opening can be made in the peritoneum of an anaesthetised patient to enable the insertion of a laparoscopic tool to assist in laparoscopic surgery after an initial umbilical opening has been made comprising the steps of:

1) inflating the abdomen via the umbilical opening or otherwise lifting the abdomen,

2) selecting an elongate tool having a trocar support ring at its leading end,

3) introducing the ring ahead of the tool through the umbilical opening, and

4) pushing the tool in a forward sense so as to press the ring against the peritoneum to deform and locally stretch the skin, and thereafter viewing the deformation externally.

The method may further comprise the step of locating the pointed end of a trocar centrally of the deformation of the abdominal wall and forcing the trocar through the area of stretched skin so that the tip of the trocar penetrates the skin and is received in the support ring.

The method may also involve the step of removing the trocar and inserting a tool through the incision formed by the trocar.

According to another aspect of the invention there is provided a method of identifying a position at which an opening can be made in the peritoneum of a patient preparatory to the insertion of a trocar to make the opening during surgery comprising the steps of:

1) anaesthetising the patient,

2) forming an umbilical opening in manner known per se,

3) inflating the abdomen with gas via the umbilical opening, or otherwise lifting the abdomen,

4) inserting through the opening an elongate tool having a loop or ring at its leading end, the loop or ring extending from the leading end of the tool with the plane of the loop or ring non-parallel to the axis of the tool, and

5) pushing the loop or ring against the internal surface of the abdomen wall so as to locally distend the skin and create an area of stretched skin bounded by the outline of the loop or ring, which is visible externally of the abdomen, to define an area thgrough which a trocar can be inserted to make the opening.

As before the method may also include the step of locating the pointed end of a trocar centrally of the deformation of the abdominal wall and forcing the trocar through the area of stretched skin so that the tip of the trocar penetrates the skin and is received in the support ring, and thereafter removing the trocar to allow a tool to be inserted.

According to a further aspect of the invention there is provided a method of identifying a position at which an opening can be made in the peritoneum of a patient preparatory to inserting a tool therethrough to assist in laparoscopic surgery, comprising the steps of:

1) anaesthetising the patient;

2) forming an umbilical opening in manner known per se;

3) inflating abdomen with gas via umbilical opening, or otherwise lifting the abdomen;

4) inserting thorough the umbilical opening a laparoscopic telescope from the leading end of which extends a shaft with a trocar support ring at the leading end thereof, whereby the ring and shaft are introduced through the umbilical opening ahead of the telescope;

5) illuminating the peritoneum on the inside of the abdomen wall to allow it to be views using the telescope, and

6) moving the telescope so as to position the ring against a region of the peritoneum through which a trocar incision can safely be made, and thereafter pushing the telescope further through the umbilical opening so as to deform the skin of the abdominal wall by the ring, so that the deformation of the skin created by the ring which indicates the position at which the trocar can be inserted, is visible from externally.

The method may further comprise the step of viewing the abdominal wall from externally without reducing the force exerted on the peritoneum by the ring, to identify the deformation therein caused by the ring, externally positioning a trocar centrally of the deformation, and forcing the trocar so as to penetrate the stretched skin, so that the tip of the trocar is received in the ring after penetration and after penetration the trocar can be removed and a laparoscopic tool can be inserted through the incision formed by the trocar.

According to a still further aspect of the invention there is provided a method of identifying a further position at which another trocar incision can be made in the peritoneum of an anaesthetised patient to allow a second laparoscopic tool to be inserted to assist laparoscopic surgery after a first trocar incision has been made in accordance with any of the aforementioned methods, comprising the further steps of:

1) after removing the trocar introducing through the first incision an elongate tool having a loop or ring at its leading end the plane of which is non parallel to the axis of the tool,

2) viewing the interior of the abdominal cavity through a laparoscopic telescope inserted through the umbilical opening to assist in positioning the loop or ring against the region of the peritoneum in which another trocar incision can safely be made, and

3) applying force to push the tool in a forward sense so as to press the loop or ring against the peritoneum and deform the latter, and thereafter viewing the abdominal skin deformation externally.

As before the method further comprises the step of locating the pointed end of a trocar centrally of the deformation externally of the abdominal wall, and forcing the trocar through the stretched ring of skin to form the second incision so that the tip of the trocar is received in the ring, and may further comprise the steps of removing the trocar and inserting the second tool through the second incision formed by the trocar.

According to another aspect of the invention there is provided an elongate tool for use in any of the aforementioned methods in which a loop or ring extends from one end of the tool with its plane non-parallel to the axis of the tool, and the loop or ring and the body of the tool are substantially rigid and non-deformable to allow the loop or ring to be pushed into contact with the internal abdomen wall and to stretch the skin of the wall to form an externally visible taut area of skin, through which a trocar can be pushed.

Preferably size of the loop or ring is selected so that it is too small to allow the trocar to pass completely therethrough.

The loop or ring may be continuous, or discontinuous so as for example to define a U or C shape.

The tool may further comprise cage or cap means to the rear of the loop or ring which is adapted in use to receive the leading end of a trocar which has been inserted through the stretched skin, thereby to prevent the trocar point from penetrating an internal organ within the abdominal cavity.

According therefore to another aspect of the invention there is provided an elongate laparoscopic instrument having a non-deformable ring or loop or cage at its foremost end so that when it is pushed against the inside of an abdominal wall in use, it deforms the wall such that when viewed externally a ring of stretched skin can be seen which defines a region of the abdominal wall through which the tip of a trocar can be inserted.

In an instrument as aforesaid which the plane of the ring or open end of the cup or cage may be angled relative to the lengthwise axis of the instrument.

In one embodiment the plane of the ring or open end of the cup or cage is generally perpendicular to the lengthwise axis of the instrument.

Where the plane of the ring is supported by a cranked arm or spokes or is angled relative to the lengthwise axis of the instrument it will be clearly visible in the field of view of a laparoscopic telescope inserted to view the abdominal cavity internally.

It is an advantage of the invention that by pushing the ring against the peritoneum, the abdominal wall is moved away from the intra-abdominal viscera and organs. In addition the pressure on the abdomen wall not only stretches but also thereby reduces the thickness of the stretched abdominal wall, thus facilitating trocar entry through the centre of the stretched ring of skin.

Typically the ring is circular and its internal diameter is either smaller than the maximum diameter of most trocars used commercially, or is smaller than the selected trocar in combination with the abdominal wall it is to penetrate, so that by pushing the ring outwardly against the peritoneum from internally, the trocar (which is pushed inwardly in the opposite sense) will be prevented from penetrating significantly into the abdominal cavity.

By pushing the loop or ring against the peritoneum, the force applied by the trocar on the skin is now more effective because it is concentrated over a smaller surface area due to the resistance presented to the surrounding skin by the presence of the ring, loop, cup or cage rim. As previously described, once the trocar tip has penetrated the abdominal wall it enters the loop, ring, cup or cage but by appropriately selecting the size of the loop or ring, the trocar can be prevented from passing completely therethrough, so that by pushing such a loop or ring against the peritoneum, the trocar tip is prevented from penetrating significantly into the abdominal cavity, and if a cage or cup is employed the tip is wholly contained therein and prevented from damaging internal organs.

### DESCRIPTION OF THE DRAWINGS

FIG. 1 is a trocar support tool as seen from the top.

FIG. 2 shows the trocar support tool from the side.

FIG. 3 shows a view of the trocar support ring 1 as seen through the telescope.

FIG. 4 shows the trocar support tool attached to a laparoscopic telescope.

FIG. 5 shows a trocar support tool attached to a laparoscopic tool.

FIG. 6 shows a schematic representation of the peritoneum distortion preceding a trocar insertion as seen from a laparoscopic telescope without the use of the invention.

FIG. 7 shows the tip of the trocar breaking through the peritoneum (again without the use of the invention).

FIG. 8 is a schematic representation of a trocar insertion, with a trocar support positioned in accordance with the invention.

FIG. 9 shows the trocar after it has penetrated the peritoneal lining of the abdominal wall and entered the ring of the trocar support.

FIG. 10 illustrates what will be seen externally if a ring is pushed against the inside of the abdomen wall, and

FIGS. 11(a)-11(f) illustrate different forms of ring or loop with and without a cage or cup to contain the trocar tip after penetration of the peritoneum by the latter.

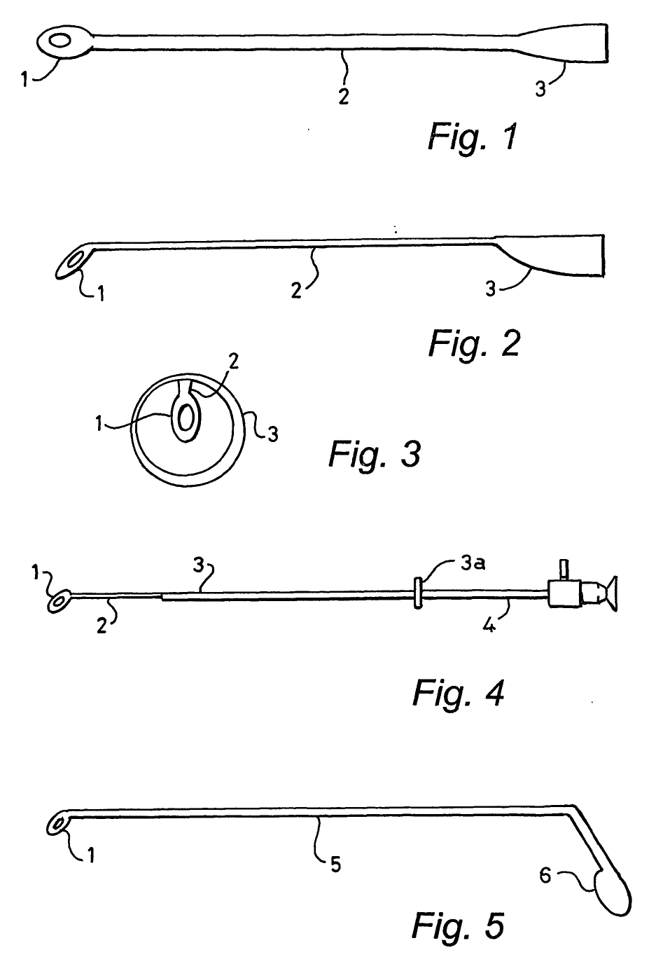

### DESCRIPTION OF THE FIGURES

Reference numeral 1 identifies a ring which is to be positioned on and pressed against the inside of the abdominal wall to identify by locally stretching and deforming the wall, where a trocar can be safely inserted, and serve as a trocar support during the trocar insertion. Reference numeral 2 identifies the main shaft to which the trocar support ring is attached, and reference numeral 3 identifies an enlarged opposite end of the shaft, which is thereby adapted to be fitted to one end of the laparoscopic telescope.

The ring 1 is angled relative to the shaft 2 so that if fitted to a laparoscopic telescope, the ring is visible in the field of view of the telescope. This enables the ring to be seen as it as it is positioned against the peritoneum to define the region of the abdomen wall through which a trocar can be inserted.

The view of FIG. 3 shows how the ring appears in the field of view of the telescope.

A complete assembly of the tool and a telescope is shown in FIG. 4, where the telescope (of known design) is denoted by reference numeral 4. The shaft 2, 3 is prevented from sliding off the telescope by means of a clamp or stop device 3a, which is not shown in the other Figures.

Alternatively as shown in FIG. 5 the ring 1 may be attached to a shaft 5 of a laparoscopic tool having a handle 6, which can be inserted in place of a tool attached to a telescope, so as to be used without, or in conjunction with, a telescope, as desired.

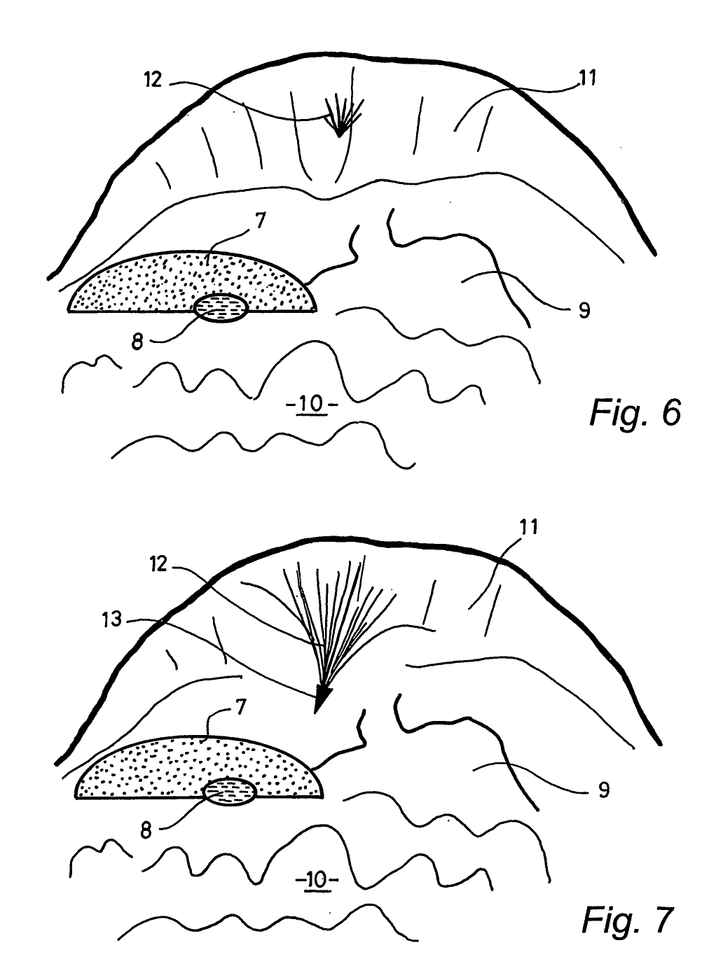

FIG. 6 shows the view as would be seen via a telescope as a trocar penetrates into the abdominal cavity. The telescope is assumed to enter via the umbilicus and to be looking up towards the head of the patient. The internal abdominal organs and structures are labelled with reference numbers as follows:

The gallbladder (8) lies in front of the liver (7).

The stomach (9) lies above the small intestine (10).

The peritoneal lining on the inside of the abdominal wall is denoted by (11) and this is seen being deformed at (12) to form a wigwam or tent-like shape by the tip of a trocar pressed against the skin from outside.

FIG. 7 shows schematically what would be seen via a telescope positioned as described in relation to FIG. 6, after the tip (13) of the trocar has just penetrated the apex of the tented lining (12) of the peritoneum.

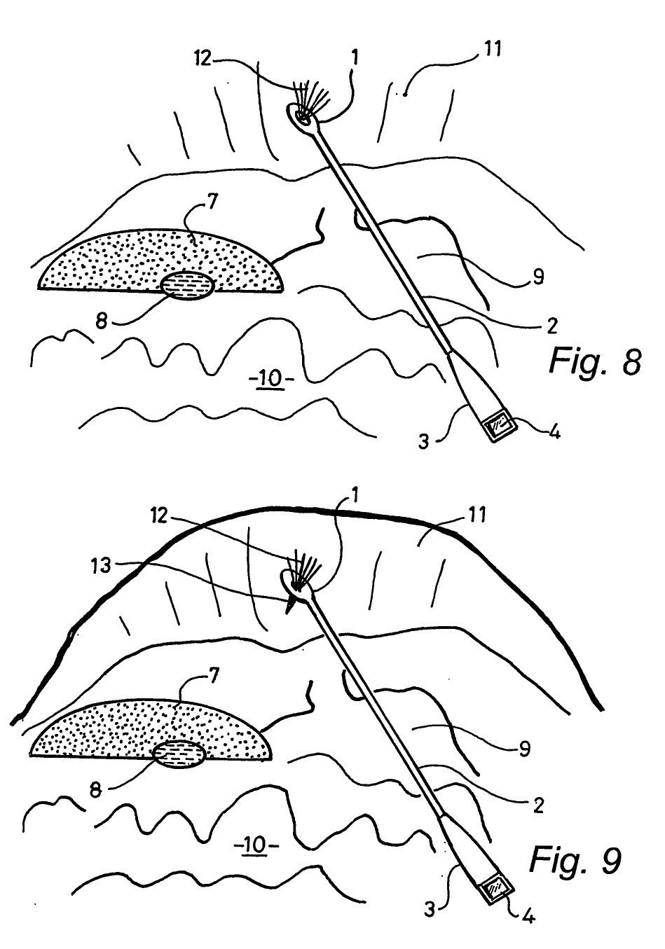

FIGS. 8 and 9 show how by pressing the ring (1) against the peritoneum (11) so that the latter is stretched so as to indicate externally where to insert a trocar, and after penetration the trocar tip enters the support during the tenting step, and thereafter is prevented from excessive penetration by the presence of the ring. In FIGS. 8 and 9, the ring 1 is shown at the end of a shaft 2 attached at 3 to the end of the telescope 4. However, it is to be understood that the ring 1 may instead be carried at the end of a shaft 5 of a tool having a handle 6 such as shown in FIG. 5, and a telescope may be employed if desired for example by inserting the telescope through another abdominal opening.

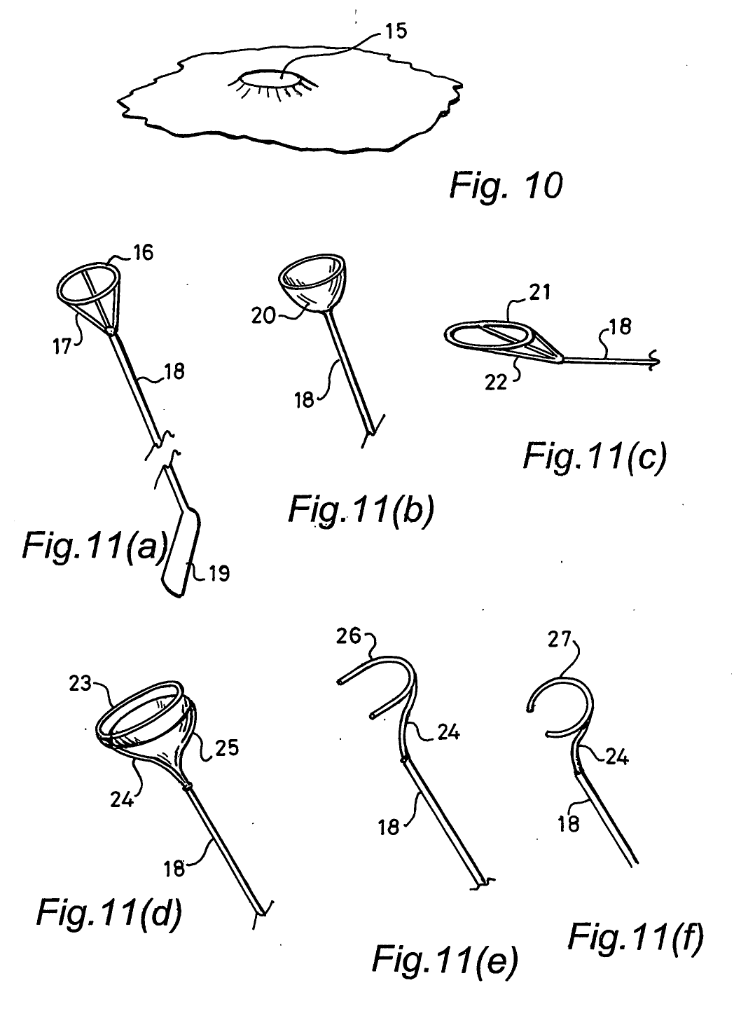

FIG. 10 illustrates the action of the support ring 1 and how it stretches skin of the abdomen and defines the region 15 which will be visible externally to serve as an indication as to where the trocar tip can be inserted.

As the externally applied force exerted through the trocar is increased, the trocar tip penetrates the abdomen wall and peritoneum lining, centrally of the circle of stretched skin 15. By carefully selecting the size of the ring 1, so that the trocar will not pass completely therthrough and the trocar is prevented by the ring 1 from entering deep into the abdominal cavity after the peritoneum has been perforated. Thus the ring will not only serve to identify (by stretching and deforming the abdominal skin) where a trocar can be inserted, but will (by appropriate choice of size) prevent the trocar tip from entering too far into the abdominal cavity.

FIG. 11 shows by way of example only various forms of support ring or cage or cup which can be mounted at the leading end of the tool.

Thus in FIG. 11(a) ring 16 is carried by four converging spokes 17 which extend from the end of the shaft 18 of the tool, which has a handle 19. The plane of the rig is generally perpendicular to the lengthwise axis of the shaft 18.

In FIG. 11(b) the ring and spokes are replaced by a cup 20. The rim of the cup lies in a plane which is generally perpendicular to the lengthwise axis of the shaft 18.

In FIG. 11(c) the spokes 22 are of differing length so that the ring 21 extends at an acute angle to the lengthwise axis of the shaft 18.

Although not shown the cup 20 may be deformed so that the rim also lies in a plane which is at an acute angle to the lengthwise axis of the shaft 18.

In FIG. 11(d) a ring 23 is carried at the end of a cranked support arm 24 extending from or forming the leading end of the shaft 18 and a cup 25 is attached to the arm 24 to receive the trocar tip, after penetrating the skin stretched by the ring 21.

FIGS. 11(e) and 11(f) show discontinuous rings in which ring 26 of FIG. 11(e) is U-shaped and ring 27 of FIG. 11(f) is C-shaped. As shown the discontinuous rings lie in a plane which is perpendicular to the lengthwise axis of the shaft 18 but it is to be understood either could be angled as is the ring 21 of FIG. 11(c) and either can include a cage of spokes such as 17 or 22 or a cup which may be located below the ring as is 25 below 23 in FIG. 11(d) or a cutaway cup having a U or C shaped rim may be employed in place of the ring 26 or 17.

In each case where a cranked support arm is shown, it is to be understood that a straight arm may be employed in lieu.

1. A method of identifying a position at which an opening can be made in the peritoneum of an anaesthetised patient to enable the insertion of a laparoscopic tool to assist in laparoscopic surgery after an initial umbilical opening has been made comprising the steps of:
1) inflating the abdomen via the umbilical opening or otherwise lifting the abdomen,
2) selecting an elongate tool having a trocar support ring at its leading end,
3) introducing the ring ahead of the tool through the umbilical opening, and
4) pushing the tool in a forward sense so as to press the ring against the peritoneum to deform and locally stretch the skin, and thereafter viewing the deformation externally.

2. A method as claimed in claim 1 further comprising the step of locating the pointed end of a trocar centrally of the deformation of the abdominal wall and forcing the trocar through the area of stretched skin so that the tip of the trocar penetrates the skin and is received in the support ring.

3. A method as claimed in claim 2 further comprising the step of removing the trocar and inserting a tool through the incision formed by the trocar.

4. A method of identifying a position at which an opening can be made in the peritoneum of a patient preparatory to the insertion of a trocar to make the opening during surgery comprising the steps of:
1) anaesthetising the patient,
2) forming an umbilical opening in manner known per se,
3) inflating the abdomen with gas via the umbilical opening, or otherwise lifting the abdomen,
4) inserting through the opening an elongate tool having a loop or ring at its leading end, the loop or ring extending from the leading end of the tool with the plane of the loop or ring non-parallel to the axis of the tool,
5) pushing the loop or ring against the internal surface of the abdomen wall so as to locally distend the skin and create an area of stretched skin bounded by the outline of the loop or ring, which is visible externally of the abdomen, to define an area through which a trocar can be inserted to make the opening.

5. A method as claimed in claim 4 further comprising the step of locating the pointed end of a trocar centrally of the deformation of the abdominal wall and forcing the trocar through the area of stretched skin so that the tip of the trocar penetrates the skin and is received in the support ring.

6. A method of identifying a position at which an opening can be made in the peritoneum of a patient preparatory to inserting a tool therethrough to assist in laparoscopic surgery, comprising the steps of:
1) anaesthetising the patient;
2) forming an umbilical opening in manner known per se;
3) inflating abdomen with gas via umbilical opening, or otherwise lifting the abdomen;
4) inserting thorough the umbilical opening a laparoscopic telescope from the leading end of which extends a shaft with a trocar support ring at the leading end thereof, whereby the ring and shaft are introduced through the umbilical opening ahead of the telescope;
5) illuminating the peritoneum on the inside of the abdomen wall to allow it to be viewed using the telescope, and
6) moving the telescope so as to position the ring against a region of the peritoneum through which a trocar incision can safely be made, and thereafter pushing the telescope further through the umbilical opening so as to deform the skin of the abdominal wall by the ring, so that the deformation of the skin created by the ring which indicates the position at which the trocar can be inserted, is visible from externally.

7. A method as claimed in claim 6 further comprising the step of viewing the abdominal wall from externally without reducing the force exerted on the peritoneum by the ring, to identify the deformation therein caused by the ring, externally positioning a trocar centrally of the deformation, and forcing the trocar so as to penetrate the stretched skin, so that the tip of the trocar is received in the ring after penetration.

8. A method as claimed in claim 7 wherein after penetration the trocar is removed and a laparoscopic tool is inserted through the incision formed by the trocar.

9. A method of identifying a further position at which another trocar incision can be made in the peritoneum of an anaesthetised patient to allow a second laparoscopic tool to be inserted to assist laparoscopic surgery after a first trocar incision has been made in accordance with the method of claim 2 comprising the further steps of:
1) after removing the trocar introducing through the first incision an elongate tool having a loop or ring at its leading end the plane of which is non parallel to the axis of the tool,
2) viewing the interior of the abdominal cavity through a laparoscopic telescope inserted through the umbilical opening to assist in positioning the loop or ring against the region of the peritoneum in which another trocar incision can safely be made, and
3) applying force to push the tool in a forward sense so as to press the loop or ring against the peritoneum and deform the latter, and thereafter viewing the abdominal skin deformation externally.

10. A method as claimed in claim 9 further comprising the step of locating the pointed end of a trocar centrally of the deformation externally of the abdominal wall, and forcing the trocar through the stretched ring of skin to form the second incision so that the tip of the trocar is received in the ring.

11. A method as claimed in claim 10 further comprising the steps of removing the trocar and inserting the second tool through the second incision formed by the trocar.

12. An elongate tool for use in the method of claim 1 in which a loop or ring extends from one end of the body of the tool with its plane non-parallel to the axis of the tool, and the loop or ring and the body of the tool are substantially rigid and non-deformable and arranged so as to allow the tool to be used to push the loop or ring into contact with the internal abdomen wall and to stretch the skin of the wall to form an externally visible taut area of skin bounded by the outline of the loop or ring, through which a trocar can be pushed.

13. A tool as claimed in claim 12 wherein the size of the loop or ring is selected so that it is too small to allow a trocar to pass completely therethrough.

14. A tool as claimed in claim 12 further comprising cage or cup means to the rear of the loop or ring, which cage or cup means is adapted in use to receive the leading end of a trocar which has been inserted through the stretched skin, thereby to prevent the trocar point from penetrating an internal organ within the abdominal cavity.

15. A tool as claimed in claim 12 wherein the loop or ring is discontinuous and defines a U or C shape.

16. An elongate laparoscopic instrument having a substantially rigid body with a non-deformable ring or loop or cage at its foremost end arranged so that the instrument can be used to push the ring or loop or cage against the inside of an abdominal wall, deforming the wall such that when viewed externally a ring of stretched skin can be seen which defines a region of the abdominal wall through which the tip of a trocar can be inserted.

17. An instrument as claimed in claim 16 in which the plane of the ring or open end of the cup or cage is angled relative to the lengthwise axis of the instrument.

18. An instrument as claimed in claim 16 wherein the plane of the ring or open end of the cup or cage is generally perpendicular to the lengthwise axis of the instrument.
